SEQUENCE LISTING (16 sequences)

SEQ ID NO: 1 - DNA, 20 bp, Homo sapiens
gatgtggagtgcctgatgtg

SEQ ID NO: 2 - DNA, 20 bp, Homo sapiens
tgcttgaccctcagagacct

SEQ ID NO: 3 - DNA, 20 bp, Homo sapiens
tctgcaaggttatcccaagg

SEQ ID NO: 4 - DNA, 20 bp, Homo sapiens
tattcctggaaaggcacctg

SEQ ID NO: 5 - DNA, 20 bp, Homo sapiens
ggagcctctcagtcatggag

SEQ ID NO: 6 - DNA, 20 bp, Homo sapiens
ttgagctggactcattgtcg

SEQ ID NO: 7 - DNA, 20 bp, Homo sapiens
tgaaggtcggtgtgaacgga

SEQ ID NO: 8 - DNA, 23 bp, Homo sapiens
catgtagccatgaggtccaccac

SEQ ID NO: 9 - DNA, 20 bp, Homo sapiens
cactgtccacccctcagagc

SEQ ID NO: 10 - DNA, 18 bp, Homo sapiens
cacttgtcggcgataagg

SEQ ID NO: 11 - DNA, 20 bp, Homo sapiens
aattccgacctcgtcatcag

SEQ ID NO: 12 - DNA, 20 bp, Homo sapiens
tgcagttttccagcaatgag

SEQ ID NO: 13 - DNA, 20 bp, Homo sapiens
tgatccacacacgttggtct

SEQ ID NO: 14 - DNA, 20 bp, Homo sapiens
tttgagttgcttgcaggatg

SEQ ID NO: 15 - DNA, 20 bp, Homo sapiens
gagtcaacggatttggtcgt

SEQ ID NO: 16 - DNA, 20 bp, Homo sapiens
gacaagcttcccgttctcag

(19) United States
(12) Patent Application Publication
(10) Pub. No.: US 2012/0322758 A1
(43) Pub. Date: Dec. 20, 2012

(54) ANTI-WRINKLE COSMETIC COMPOSITION FOR COMPRISING 3-5 kDa CHITOOLIGOSACCHARIDES

(75) Inventors: Se Kwon Kim, Busan (KR); Chang Suk Kong, Busan (KR); Soon Sun Bak, Busan (KR); Jung Ae Kim, Busan (KR); Byul Nim Ahn, Busan (KR)

(73) Assignee: PUKYONG NATIONAL UNIVERSITY INDUSTRY-UNIVERSITY CORPORATION FOUNDATION, Busan (KR)

(21) Appl. No.: 13/523,350

(22) Filed: Jun. 14, 2012

(30) Foreign Application Priority Data
Jun. 14, 2011 (KR) 10-2011-0057678

(51) Int. Cl. A61K 8/73; A61Q 19/08; C08B 37/08

(52) U.S. Cl. 514/55; 536/20

(57) ABSTRACT

The present invention relates to an anti-wrinkle cosmetic composition comprising chitooligosaccharide as an effective component, more specifically, the cosmetic composition to inhibit collagen degradation induced by UV-A irradiation.

### TECHNICAL FIELD

The present invention relates to an anti-wrinkle cosmetic composition comprising chitooligosaccharide as an effective component, more specifically, the cosmetic composition for inhibiting collagen degradation induced by UV-A irradiation.

### BACKGROUND ART

Skin aging is involved in multiple processes in a consequence of the passage of time (chronologic or natural aging) and damage due to ultraviolet (UV) light from the sun (photoaging). UV exposure causes changes in the internal structure of the skin such as dysplasia of the keratinocytes in the epidermis and deterioration of the extracellular matrix component in the dermis. These changes due to photoaging in the skin appear as loss of elasticity, sagging of the skin, wrinkling, dyspigmentation and accumulation of precancerous lesions. Photoaging is partially caused by damage to skin connective tissue by reduced collagen synthesis and by increased collagen degrading matrix metalloproteinases (MMPs) expression. In particular, the levels of collagen degrading MMPs are known to rise in fibroblasts as a function of age and oxidative stress is also associated with photoaging of skin.

Collagen is a main dermal component of extracellular matrix and degeneration of collagen caused by UV irradiation is a key factor in dermal alteration. UV light can be divided into UV-A (400-315 nm), UV-B (315-280 nm) and UV-C (280-100 nm) according to the wave length. Most of all, UV-A region possesses strong skin infiltration ability and 98.7% of UV reach as UV-A in the earth's surface. Excessive UV-A exposure results in not only wrinkle formation but also skin cancer. Therefore, inhibition of collagen degrading MMPs expression by increasing collagen synthesis and blockage of UV exposure will enable the development of improved strategies to repair photodamaged skin.

Chitooligosaccharide (COS) is a water soluble chitosan made from chitin or chitosan by chemical or enzymatic hydrolysis method. COS has wide usage due to lots of advantages such as water solubility, biocompatibility, and biodegradability. It can be also used as cosmetic materials as it has anti-aging, whitening, and moisturizing function. In addition, previous studies reported that COS has anti-microbial, anti-tumoral, anti-fungal, anti-viral, and anti-oxidative activities according to its molecular size.

Relating to COS, Korean Patent No. 10-0861415 discloses a cosmetic composition containing myrrh, Sanguisorba officinalis, acanthopanax, chinese pepper, Eriobotrya japonica Lindley, sophora extract, and COS. A cosmetic composition comprising water-soluble chitosan with high molecular weight 10 kDa to 1,500 kDa is disclosed in Korean Patent Publication No.

10-2001-0060436 as well. Nevertheless, effect of 3-5 kDa COS on UV-A induced collagen degradation in dermis cells has not been investigated to date.

### BRIEF DESCRIPTION OF THE DRAWINGS

FIG. 1 shows effect of 3-5 kDa COS on UV protection obtained from UV absorption spectrum.

FIG. 2 shows effect of 3-5 kDa COS on collagen degrading MMPs secretion in human dermal fibroblasts exposed to 6 J/cm² of UV-A irradiation. The levels of collagen degrading MMPs secretion such as MMP-1 (A), MMP-8 (B) and MMP-13 (C) were determined by ELISA assay. Means with the different letters are significantly different (p<0.05) between samples and blank or control at each concentration. Bl: without UV-A irradiation, Con: only UV-A irradiated group.

FIG. 3 shows effect of 3-5 kDa COS on the levels of collagen degrading MMPs in UV-A irradiated HDF cells. Gene (A) and protein (B) expression levels were examined by RT-PCR and Western blot analysis, respectively.

FIG. 4 shows effect of 3-5 kDa COS on collagen synthesis in UV-A irradiated HDF cells.

### DESCRIPTION OF THE PREFERRED EMBODIMENT

### Technical Problem

The object of the present invention is to provide an anti-wrinkle cosmetic composition comprising chitooligosaccharide as an effective component.

### Technical Solution

The above object of the present invention was achieved by measuring UV absorption spectrum of 3-5 kDa COS and determining the effect of 3-5 kDa COS on collagen degrading MMPs expression and collagen synthesis in UV-A irradiated HDF cells by RT-PCR and Western blot analysis.

### Advantageous Effects

The present invention identifies the protective effect of 3-5 kDa COS on UV-A induced collagen degradation, therefore providing a highly effective anti-wrinkle cosmetic composition using the same.

### BEST MODE FOR THE INVENTION

The present invention provides an anti-wrinkle cosmetic composition comprising 3-5 kDa COS (chitooligosaccharide) as an effective component for preventing or treating collagen degradation due to UV-A.

The cosmetic composition in the present invention can include carrier and conventional supplement such as antioxidant, stabilizer, solubilizer, vitamin, pigment, and perfume.

The cosmetic composition of the present invention can be prepared as any formulation by the well-known method in the art, for example, solution, suspension, emulsion, paste, gel, cream, lotion, powder, soap, surfactant-containing cleansing , oil, powdered foundation, emulsion foundation, a wax foundation, spray, etc, however is not limited thereto. More specifically, the formulation can be prepared as skin softener, skin toner, nutrition lotion, nutrient cream, massage cream, essence, eye cream, cleansing cream, cleansing foam, cleansing water, pack, spray or powder.

In case a composition of the present invention is paste, cream or gel, a carrier component can consist of animal oil, vegetable oil, wax, paraffin, starch, traganth, cellulose derivative, polyethylene glycol, silicone, bentonite, silica, talc, zinc oxide, etc.

In case a composition of the present invention is powder or spray, the carrier component can include lactose, talc, silica, aluminum hydroxide, calcium silicate or polyamide powder, and spray can additionally comprise propellant such as chlorofluorohydrocarbon, propane/butane or dimethyl ether.

In case a composition of the present invention is solution or emulsion, the carrier component includes solvent, solubilizer or emulsion agent.

In case a composition of the present invention is suspension, the carrier component can contain liquid diluents such as water, ethanol or propylene glycol, suspending agents such as polyoxyethylene sorbitol esters, microcrystalline cellulose, and so on.

In case a formulation of the present invention is surfactant-containing cleansing, the carrier component can comprise aliphatic alcohol sulfate, aliphatic alcohol ether sulfate, etc.

Hereinafter, the present invention will be described by the following examples in more detail. However, such examples are only to illustrate the invention and they do not restrict the present invention.

Data were expressed as mean±SD (n=3) and analyzed using the analysis of variance (ANOVA) procedure of Statistical Analysis System (SAS v9.1, SAS Institute Inc., Cary, NC,

USA). Significant differences between treatment means were determined using Duncan's multiple range tests at the p<0.05 level.

### Example 1

### Cell Culture

The 3-5 kDa COS (chitooligosaccharide) was donated by Kitto Life Co. (Seoul, Korea). Human dermal fibroblast (HDF) cells isolated from the dermis of adult skin were purchased from Promo cell (Heidelberg, Germany). HDF cells were grown in Dulbecco's modified Eagle's medium (DMEM, Gibco-BRL, Gaithersburg, Md., USA) containing 10% fetal bovine serum (FBS), 2 mM glutamine, and 100 μg/ml penicillin-streptomycin (Gibco-BRL, Gaithersburg, Md., USA) at 37° C. humidified atmosphere of 5% CO₂. The cells (passage 2) were maintained for 6 additional passages and sub-cultured for experiment at about 90-95% confluence by detaching with trypsin-EDTA solution.

### Example 2

### UV Absorption Spectrum of 3-5 kDa COS

To obtain UV absorption ability of 3-5 kDa COS, absorption spectrum was analyzed by spectrophotometer. Briefly, 3-5 kDa COS was dissolved in D.W. at the concentration of 1 mg/ml, followed by measuring UV spectra between 190 nm and 500 nm using UV-Vis spectrophotometer (Varian, Walnut Creek, Calif., USA) with a cuvette of 1 cm path length at room temperature. Each spectrum was signal-averaged at least three times with a bandwidth of 1.0 nm and a resolution of 0.5 nm at a scan rate of 600 nm/min.

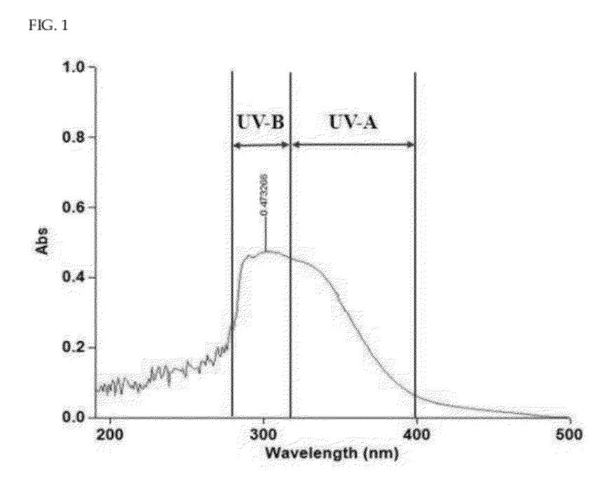

As shown in FIG. 1, 3-5 kDa COS absorbed not only UV-A but also UV-B range. Therefore, COS might reduce photodamage of skin by blocking the UV light. Based on the result, it was confirmed that 3-5 kDa COS can be developed as a sun protective cosmetic material thereby absorb UV light.

### Example 3

### UV-A Irradiation

HDF cells were seeded in 24-well plate at a density of 1×10⁵ cells/well with DMEM containing 10% FBS, 2 mM glutamine, and 100 μg/ml penicillin-streptomycin at 37 ° C. under 5% CO₂ atmosphere. After incubation for 24 h, the cells were exposed to UV-A energy at 6 J/cm² (312 nm UV-A light source, Bio-Sun lamp, Vilber Lourmat, Marine, France) in 200 μl of phosphate buffered saline (PBS) to each well, followed by incubation in serum-free DMEM.

### Example 4

### Determination of Collagen Degrading Mmps Level by Enzyme-Linked Immunosorbent Assay (ELISA)

Collagens are known as main component of the skin and degraded by collagenase MMPs. Among MMPs, MMP-1, MMP-8, and MMP-13 are family of collagenases which are capable of degrading triple-helical fibrillar collagens. Loss of skin collagen is resulted in wrinkle formation. Therefore, inhibitory effect of 3-5 kDa COS on collagenase MMP secretions from the UV-A irradiated HDF cells was determined using ELISA assay as follows:

HDF cells were incubated with the different concentration of 3-5 kDa COS in the absence of FBS, followed by UV-A irradiation. After 24 h, the cells were centrifuged and then the supernatants were collected for MMPs assay. The assay was designed to assess and quantify the levels of MMP-1, MMP-8, and MMP-13 using Biotrak^{TM} ELISA kits (Amersham Pharmacia Biosciences) according to the manufacturer's instructions. Briefly, 100 μl of each standard and samples were added in triplicate into the appropriate wells. After incubation for 2 h at room temperature, the wells were emptied and washed 4 times with 400 μl of wash buffer. Then 100 μl of peroxidase conjugate was added into the wells for 1 h. After incubation, TMB substrate (100 μl) was added to each well and incubated for 30 min at room temperature, followed by adding 100 μl of stop solution to all wells including blank wells. The absorbance was determined at 410 nm using the above microplate reader. The standard curves of MMP-1, MMP-8, and MMP-13 were established by plotting the pg/ml concentrations versus absorbance values of standard wells. It was used to quantify the amount of MMP-1, MMP-8, and MMP-13 secreted from the cells.

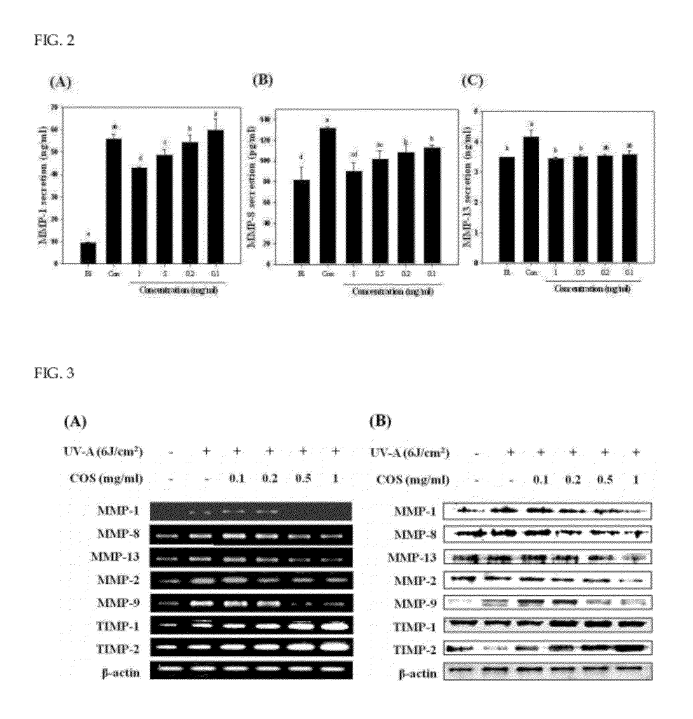

As shown in FIG. 2, MMP-1, MMP-8, and MMP-13 secretions were reduced in the presence of 3-5 kDa COS in a dose-dependent manner. These results confirmed that 3-5 kDa COS exerted adequate protective effect on collagen degradation by UV-A irradiation.

### EXAMPLE 5

### Effect of 3-5 kDa COS on Collagen Degrading Mmps Expression and Collagen Synthesis in UV-A Irradiated HDF Cells

### Example 5-1:

### Reverse Transcription Polymerase Chain Reaction (RT-PCR) Analysis

Total cellular RNA was isolated using Trizol reagent (Invitrogen Co., CA, USA). Two micrograms of isolated RNA were reverse transcribed into cDNA using oligo-(dT) primer (Promega, Madison, Wisc., USA). Target cDNA was amplified with specific primers as follows: forward 5′-GAT-GTG-GAG-TGC-CTG-ATG-TG-3′ (SEQ ID NO:1) and reverse 5′-TGC-TTG-ACC-CTC-AGA-GAC-CT-3′ (SEQ ID NO:2) for MMP-1; forward 5′-TCT-GCA-AGG-TTA-TCC-CAA-GG-3′ (SEQ ID NO:3) and reverse 5′-TAT-TCC-TGG-AAA-GGC-ACC-TG-3′ (SEQ ID NO:4) for MMP-8; forward 5′-GGA-GCC-TCT-CAG-TCA-TGG-AG-3′ (SEQ ID NO:5) and reverse 5′-TTG-AGC-TGG-ACT-CAT-TGT-CG-3′ (SEQ ID NO:6) for MMP-13; forward 5′-TGA-AGG-TCG-GTG-TGA-ACG-GA-3′ (SEQ ID NO:7) and reverse 5′-CAT-GTA-GCC-ATG-AGG-TCC-ACC-AC-3′ (SEQ ID NO:8) for MMP-2; forward 5′-CAC-TGT-CCA-CCC-CTC-AGA-GC-3′ (SEQ ID NO:9) and reverse 5′-CAC-TTG-TCG-GCG-ATA-AGG-3′ (SEQ ID NO:10) for MMP-9; forward 5′-AAT-TCC-GAC-CTC-GTC-ATC-AG-3′ (SEQ ID NO:11) and reverse 5′-TGC-AGT-TTT-CCA-GCA-ATG-AG-3′ (SEQ ID NO:12) for TIMP-1; forward 5′-TGA-TCC-ACA-CAC-GTT-GGT-CT-3′ (SEQ ID NO:13) and reverse 5′-TTT-GAG-TTG-CTT-GCA-GGA-TG-3′ (SEQ ID NO:14) for TIMP-2; forward 5′-GAG-TCA-ACG-GAT-TTG-GTC-GT-3′ (SEQ ID NO:15) and reverse 5′-GAC-AAG-CTT-CCC-GTT-CTC-AG-3′ (SEQ ID NO:16) for β-actin. The amplification was carried out for 35 cycles at 95° C. for 45 sec, 60 ° C. for 50 sec, and 72° C. for 60 sec, followed by extension process proceeded consecutively at 72 ° C. for 5 min.

PCR products were separated on 1.5% agarose gel for 10 min at 100 V by electrophoresis. Gels were stained with 1 mg/ml of EtBr and photographed by UV illumination using AlphaEase® gel image analysis software (Alpha Innotech., San Leandro, Calif., USA).

### Example 5-2

### Western Blot Analysis

Whole cells were lysed in RIPA buffer (Sigma-Aldrich Corp., St. Louis, USA). After centrifugation, total protein amount of cell lysates was determined using Lowry method (BioRad Laboratories, Hercules, Calif.). Aliquot of supernatant containing equal amounts of proteins (15 μg) was electrophoresed on 10% or 12% SDS-PAGE gels, transferred onto a nitrocellulose membrane (Amersham Pharmacia Biotech., England, UK), blocked with 5% skim milk in TBS containing 0.1% Tween 20 (TBS-T) for at least 1 h, followed by hybridization with the primary antibodies (Santa Cruz Biotechnology Inc., Calif., USA). All primary monoclonal antibodies were diluted with TBS-T at 1:1000 ratio. Bound antibodies were detected by horseradish peroxidase-conjugated secondary antibodies for 1 h at room temperature and the immunoreactive proteins were detected using a chemiluminescent ECL assay kit (Amersham Pharmacia Biosciences, England, UK) according to the manufacturer's instructions. Western blot bands were visualized using a LAS3000® Luminescent image analyzer (Fujifilm Life Science, Tokyo, Japan)

### Example 5-3

### Effect of 3-5 kDa COS on the Levels of Collagen Degrading MMPs in UV-A Irradiated HDF Cells

FIG. 3 represents the effect of 3-5 kDa COS on collagenase MMPs expression in UV-A irradiated cells by RT-PCR and Western blot analysis. The collagenase MMP gene expression levels of MMP-1, MMP-8 and MMP-13 were reduced by 3-5 kDa COS treatment in a concentration dependent manner. In addition, the expression levels of MMP-2 and MMP-9, known as a type IV collagenase, were decreased by 3-5 kDa COS treatment compared with only UV-A irradiated cell. Moreover, these collagenase levels were regulated by TIMPs genes. The levels of TIMP-1 and TIMP-2 gene were reduced by UV-A irradiation whereas enhanced by treatment with 3-5 kDa COS. Accordingly, 3-5 kDa COS showed the protective effect on UV-A induced collagen degradation.

### Example 5-4

### Effect of 3-5 kDa COS on Collagen Synthesis in UV-A Irradiated HDF Cells

Collagens are the major components of most connective tissues. Among collagens, collagen type I, III, and IV are most abundant in human skin, and these collagens are significantly degraded by UV exposure thereby resulted in skin wrinkling

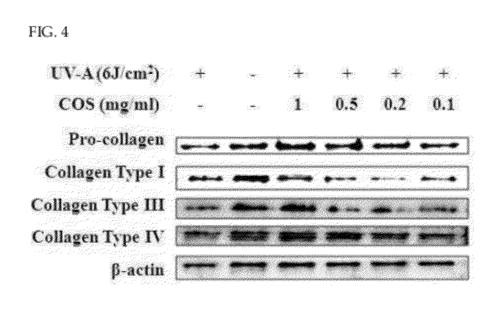

To determine whether COS can affect collagen synthesis in UV-A irradiated cells, collagen synthetic markers were examined by Western blot analysis (FIG. 4). 3-5 kDa COS Treatment increased collagen type I, III, and IV levels in a dose dependant manner. In addition, interstitial collagens are synthesized and secreted out of the cells as procollagens. Regulation of procollagens was decreased in UV-A alone exposed cells. However, the decreased cellular levels of procollagen due to UV-A exposure were increased by the presence of 3-5 kDa COS.

The above-described results indicate that 3-5 kDa COS might increase collagen production due to the reduction of collagen degrading MMP expressions.

1. An anti-wrinkle cosmetic composition comprising chitooligosaccharide as an effective component.

2. The anti-wrinkle cosmetic composition according to claim 1, wherein the chitooligosaccharide has the molecular weight of 3 to 5 kDa.

3. A composition having the inhibitory effect on UV-A induced collagen degradation, comprising 3-5 kDa chitooligosaccharide as an effective component.

4. The composition of claim 1 in the form of a paste, cream gel, powder, spray, solution, emulsion, suspension, or surfactant-containing cleanser.

5. A method of inhibiting collagen degradation in dermis cells induced by UV-A irradiation comprising applying a composition according to claim 1 to skin.

6. The method of claim 5 wherein the molecular weight if the chitoologosaccharide is 3 to 5 kDa.

7. The method of claim 5 wherein the composition is a paste, cream gel, powder, spray, solution, emulsion, suspension, or surfactant-containing cleanser.
